(19) United States
(12) Patent Application Publication
(10) Pub. No.: US 2003/0032201 A1
(43) Pub. Date: Feb. 13, 2003

(54) Method and device for electrophoresis and blotting

(76) Inventor: Robert W. Flesher, (US)

Correspondence Address: Carter, Ledyard & Hilburn, 1401 Eye Street, N.W., Suite 300, Washington, DC, 20005, US

(21) Appl. No.: 09/925,430

(22) Filed: Aug. 10, 2001

(51) Int. Cl. G01N 33/559; G01N 27/26; G01N 33/557

(52) U.S. Cl. 436/517; 204/464

(57) ABSTRACT

This application relates to a method and apparatus for the electrophoresis, blotting and detection of nucleic acids, proteins and other biological molecules using a porous polymer gel carrier and assembly. The combination of a porous polymer gel carrier and attached laminate layer facilitates the electrophoresis and subsequent blotting analysis of nucleic acids, proteins and other biological molecules. The porous polymer gel carrier also facilitates the use, handling and processing of fragile separation materials such as polyacrylimide and agarose gels. The method and apparatus also permits the convenient packaging and distribution of standardized electrophoresis and blotting products.

### BACKGROUND OF INVENTION

This application relates to method and apparatus for the electrophoresis, blotting and detection of nucleic acids, proteins and other biological molecules using a porous polymer electrophoresis gel carrier and assembly.

Electrophoresis is simple, rapid and highly sensitive. It is widely used to separate, identify and purify proteins, nucleic acids (DNA and RNA) and other complex biological molecules. In electrophoresis, molecules are mixed in a buffer solution, applied to a support material, such as a polyacrylimide or agarose gel, and are forced to move through the support material by an electric field. The support material acts as a molecular sieve by retarding, or in some cases obstructing, the movement of larger molecules while allowing smaller molecules to migrate freely. The actual movement of the molecules generally depends on the porosity of the support material, the strength of the field, the size, shape and characteristics of the sample molecules, the relative hydrophobicity of the samples, and the ionic strength and temperature of the electrophoresis buffer. The resulting differences in the rate of movement may be used to separate the molecules contained in a sample mixture.

Following electrophoresis, the separated molecules may be visualized as a series of bands separated based on their molecular size. The sample bands may be identified by radioactive labeling, autoradiography and similar techniques. The separated products may also be identified by a variety of staining techniques including staining by Coomassie blue, Amido black, ethidium bromide (“EtBr”) or various types of silver stains. In some applications, the stained gels may be dried between transparent cellophane sheets and are then suitable for notebook storage or overhead projection.

The processes of gel staining can be a relatively time consuming and painstaking tasks involving extensive handling, washing and destaining. Moreover, conventional staining and labeling techniques may also involve the use of potentially harmful or toxic chemicals or may preclude further analysis of the samples. The identification of electrophoresis gel products by autoradiography or fluorescent illumination methods may also require extensive handling and manipulation of the sample containing gel material.

The most widely used conventional eletrophoresis separation materials include polyacrylamide and agarose gels. Polyacrylimide gels provide a separation medium of varying porosity that is electrically neutral and which is transparent to optical and ultraviolet illumination. Polyacrylimide gels may be used to separate proteins and small oligonucleotides that require a relatively small gel pore size. The components of polyacrylamide gels, including acrylimide and bisacrylimide, may be extremely toxic. Agarose is a naturally occurring polysaccharide. Agarose gels may be used to separate larger macromolecules such as nucleic acids, large proteins and protein complexes. Although agarose is non-toxic, the reagents, probes, stains or buffers used with agarose based electrophoresis may also present varying degrees of hazard.

In recent years, other types of porous separation materials, including combinations of polyacrylimide and agarose, have been described and utilized for various purposes. These different types of electrophoresis separation materials have distinctive physical properties that facilitate the separation, isolation and analysis of proteins, nucleic acids and other biological molecules.

By their nature, the various types of porous separation materials, including the widely used polyacrylimide and agarose gels, can be extremely fragile or difficult to handle. The fragility of the various separation materials can complicate use, analysis and storage of electrophoresis products. Moreover, as stated above, prolonged contact with various materials used in or for electrophoresis may also be hazardous.

The identification and analysis of electrophoresis products may be further augmented by the use of various types of molecular blotting techniques. Following electrophoresis, an investigator may construct a layered sandwich consisting of buffer saturated filter paper, gel, transfer material and filter paper. The samples may then be transferred from the gel to the separation materials by capillary action. The samples may also be transferred or blotted to a separate transfer membrane by known vacuum or pressure transfer methods or a technique known as electroblotting.

The basic techniques of electrophoresis is well known and widely used. As schematically illustrated in FIG. 1a, in conventional horizontal electrophoresis, a separation material 2, such as an agorose or polyacrylimide gel, is cast between two plates 4, 5. The gel material 2 may then be immersed in a buffer containing reservoir 10 between a negative 6 and positive 8 electrode. An electric current is applied between the electrodes 6, 8 in a direction that is parallel to the horizontal plane of the gel material 2.

Following electrophoresis, the gel material 2 may be removed from the electrophoresis device and the samples subjected to analysis by molecular blotting. As schematically illustrated in FIG. 1b, in a conventional method of electroblotting, the gel material 2 together with selected transfer material 12 may be placed between a negative 6 electrode and a positive 8 electrode oriented in a direction that is perpendicular to the horizontal plane of the gel material 2. The application of the electric current between the negative 6 and positive 8 electrodes causes the sample molecules to migrate from the gel material 2 to a selected transfer material 12. Specific molecules may be identified by incubating the transfer material 12 with a labeled molecular probe or antibody. The resulting combination of target molecule and probe may then be identified and characterized by conventional detection and visualization techniques including autoradiography, fluorescence and ultraviolet illumination.

Electrophoresis and molecular blotting techniques are widely used in academic and commercial research environments. The techniques are also increasingly utilized in more routine diagnostic, commercial and forensic applications. The conventional methods and apparatus for electrophoresis and molecular blotting generally require extensive handling and manipulation of fragile and otherwise sensitive materials. These methods also require a certain degree of experience and training. Moreover, even slight variations in technique or reagents can adversely affect the consistency and reliability of results.

The present invention provides an improved method and apparatus for performing various types of electrophoresis and molecular blotting using a porous polymer matrix as an electrophoresis gel carrier and assembly.

It is an object of this invention to provide an improved method and apparatus for the sizing, separation, identification, isolation, blotting, transfer and hybridization of nucleic acids, proteins and other types of biological molecules.

It is a further object of this invention to provide a stable and functionally inert supporting matrix for electrophoresis.

It is another object of this invention to provide an improved apparatus and process for transferring and analyzing nucleic acids, proteins and other electrophoresis products.

It is yet another object of this invention to reduce the amount of manipulation and handling that is required to analyze an electrophoresis gel.

It is a further object of this invention to provide an improved method and apparatus for the storage, manipulation, and cataloging of electrophoretic analysis by manual, automatic and robotic means.

It is a further object of this invention to provide an improved method for the manual mechanical, or robotic manipulation of electrophoresis samples in high density configurations.

It is yet another object of this invention to provide an improved method and apparatus for transferring sample molecules from an electrophoresis medium to a transfer layer.

It is a further object of this invention to provide a modular gel carrier assembly that is readily adaptable for use in a variety of existing vertical and horizontal electrophoresis systems and apparatus.

It is yet another object of this invention to provide a device and method that permits cost effective and easy-to-use prepackaging of standardized electrophoresis and blotting of proteins, nucleic acids and other biological molecules.

It is a further object of the present invention that the samples isolated by electrophoresis and blotting can be readily viewed using existing illuminating methods and sources to facilitate identification of specific sample components.

These and many other features, objects and advantages of the invention will be readily apparent to one skilled in the art for and to which this invention pertains from a review of claims, the appended figures and drawings and the detailed description of the preferred embodiments when read in conjunction with the accompanying figures and drawings.

### BRIEF DESCRIPTION OF DRAWINGS

FIG. 1a is a schematic illustration of horizontal electrophoresis and FIG. 1b is a schematic illustration of electroblotting using conventional horizontal devices.

FIG. 2a and 2b are schematic illustrations of a porous polymer electrophoresis gel carrier containing parallel separation channels, a porous separation material, a laminate spacer layer and a laminate transfer layer.

FIG. 3 is a schematic illustration of how a porous polymer gel carrier may be used in the separation and identification of nucleic acid fragments using electrophoresis and blotting.

FIG. 4 is a schematic illustration of how a porous polymer gel carrier may be used in the separation and identification of proteins using electrophoresis and blotting.

FIGS. 5a and 5b are schematic side-view illustrations of an electrophoresis gel carrier assembly adapted for performing electroblotting using a conventional horizontal electrophoresis device.

### DESCRIPTION OF PREFERRED EMBODIMENTS

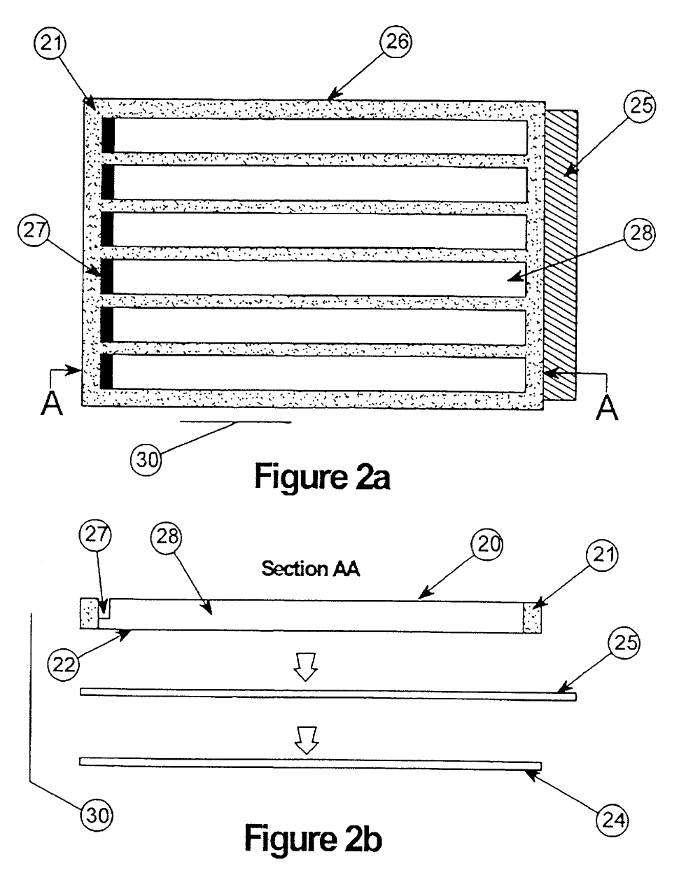

FIG. 2a is a top view of an electrophoresis gel carrier. FIG. 2b is a schematic side-view which illustrates the components of an electrophoresis gel carrier. As illustrated in FIGS. 2a and 2b, an electrophoresis gel carrier 30 may be constructed of a porous polymer such as polyethylene or a similar porous material such as reticulated polyurethane or any other type of open cell non-conductive material. The porous polymer layer 20 has opposing top 21 and bottom 22 surfaces. The side portions 26 of the porous polymer layer 20 are of equal depth around the entire perimeter of the porous polymer layer 20. The porous polymer layer 20 may contain one or more sample channels 28 that are configured to hold a selected electrophoresis separation material such as polyacrylimide, agarose or other porous separation material. Sample loading wells 27 may also be provided in each separation channel 28. The separation channels 28 are configured to pass from the top 21 to bottom 22 surface through the entire thickness of the porous polymer layer 20. The separation channel 28 or channels are oriented generally parallel to a selected axis of the horizontal planed formed by the opposing tip 21 and bottom surface of the porous polymer layer 22.

As shown in FIG. 2a, when viewed from the top, the horizontal plane of the porous polymer layer 20 is generally rectangular in shape. However, other generally orthogonal shapes or orientations may be utilized to accommodate different types of applications and apparatus. The number, depth, length and width of the separation channels 28 may also be readily tailored to accommodate the specific application.

As further illustrated in the schematic side view of FIG. 2b, a laminate layer 24 may be attached directly to the bottom surface 22 of the porous polymer layer 20 using heat, adhesive, pins or other suitable attachment means. Depending upon the intended application, the laminate layer 24 may be permanently bonded to the bottom surface 22 of the porous polymer layer 20 or may be freely removable. The laminate layer 24 may also be transparent to various means of optical and ultraviolet illumination. The laminate layer 24 may also be selectively constructed of a non-porous and non-conductive materials such as nylon, nitrocellulose, polyvinylidene fluoride (“PVDF”) or other types of non-conductive, non-porous material.

The porous polymer material used in the electrophoresis gel carrier 30 freely conducts electrical current. However, the side portions 26 of the porous polymer layer 20 and side portions of the separation channels 28 may be selectively sealed with heat or other selected sealant means. This selective sealing effectively eliminates current flow through the side portions 26 of the porous polymer layer 20 or between the individual separation channels 28.

Many widely utilized separation media, such as polyacrylimide and agarose gels, are initially prepared in a liquid form and then allowed to cool to a gel-like form. The close association of the bottom surface 22 of the porous polymer layer 20 with the laminate layer 24 permits each separation channel 28 of the porous polymer layer 20 to be selectively filled with a solution of polyacrylimide, agarose or other selected separation material and allowed to cool. The gap distance of the parallel separation channels 28 is determined by the side 26 width of the porous polymer layer 20. The gap distance may be readily adjusted to accommodate different applications and devices.

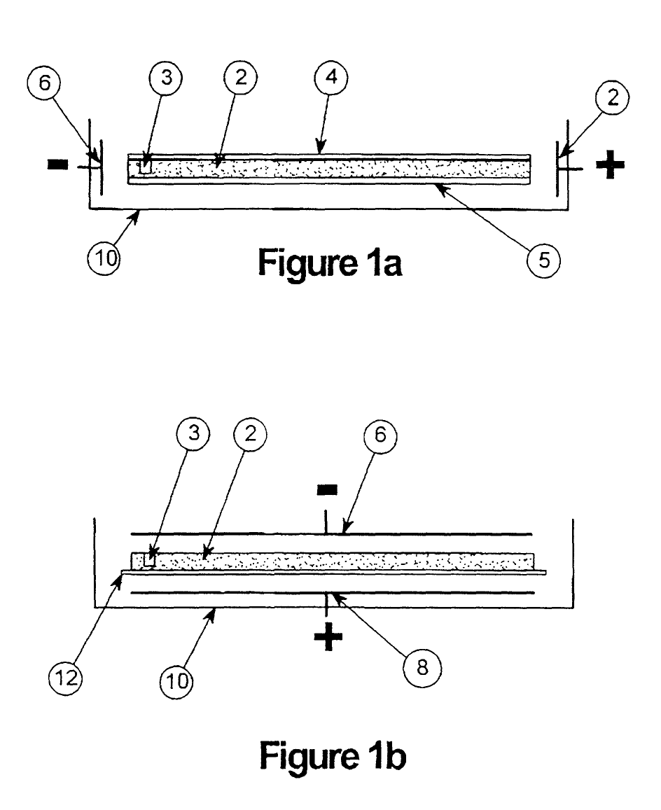

The electrophoresis gel carrier 30 is particularly suitable for use in a horizontal electrophoresis device that is similar to the device that is illustrated in FIG. 1a. In such an application, the porous polymer gel carrier may be used with or without the transparent glass or plastic cover plates 4, 5. Depending on the apparatus and application, the use of cover plates may reduce the thermal effects of the electric current. One skilled in the art will recognize that the gel carrier matrix also readily adaptable to use in a vertical orientation.

The porous polymer gel carrier can accommodate different concentrations and types of separation material in the same electrophoresis gel carrier 30. The porous polymer gel carrier 30 can also facilitate the simultaneous use of separation channels 28 containing separation materials of different composition or concentration. In one such an embodiment, each separation channel 28 may be filled with a different concentration or composition of polyacrylimide or agarose gel to facilitate the separation and screening of samples. In another embodiment, separation materials of different resolving concentrations may be used within a single gel carrier 30 to allow samples of varying molecular weight to be simultaneously analyzed. In yet another application, the individual separation channels 28 may be readily removed from the gel carrier 30 for further reaction or analysis such as polymerase chain reaction (“PCR”). In some applications, the porous polymer layer 20 of the gel carrier 30 may also be presaturated with a selected separation material.

The porous polymer material that is used in the porous polymer layer 20 of the gel carrier 30 provides a number of practical advantages to existing devices and methods. The various types of porous polymer material, such as polyethylene, reticulated polyurethane and other open cell non-conducting materials, are economical to use and are readily available in a variety of thickness and pore sizes. The porous polymer material can also be readily and economically molded, die-cut, and machined to any desired shape or form. The porous polymer materials are also non-reactive to most chemicals and are naturally hydrophobic. Moreover, electric current flows readily through the porous polymer material in any direction or orientation.

The porous polymer material is also extremely resilient and flexible. This provides a number of desirable advantages over existing methods and systems. The porous polymer layer 20 also provides stability to the otherwise fragile separation material such as agarose and polyacrylimide gels. The stability provided by the porous polymer gel carrier 30 also facilitates the labeling, manipulation, handling and storage of the separation material in the porous polymer layer 20 before, during and after electrophoresis, used conventional manual techniques or by robotic, automated or semi-automated methods and devices. The use of the porous polymer material also minimizes the need and potential for direct contact with the separation medium or related reagents.

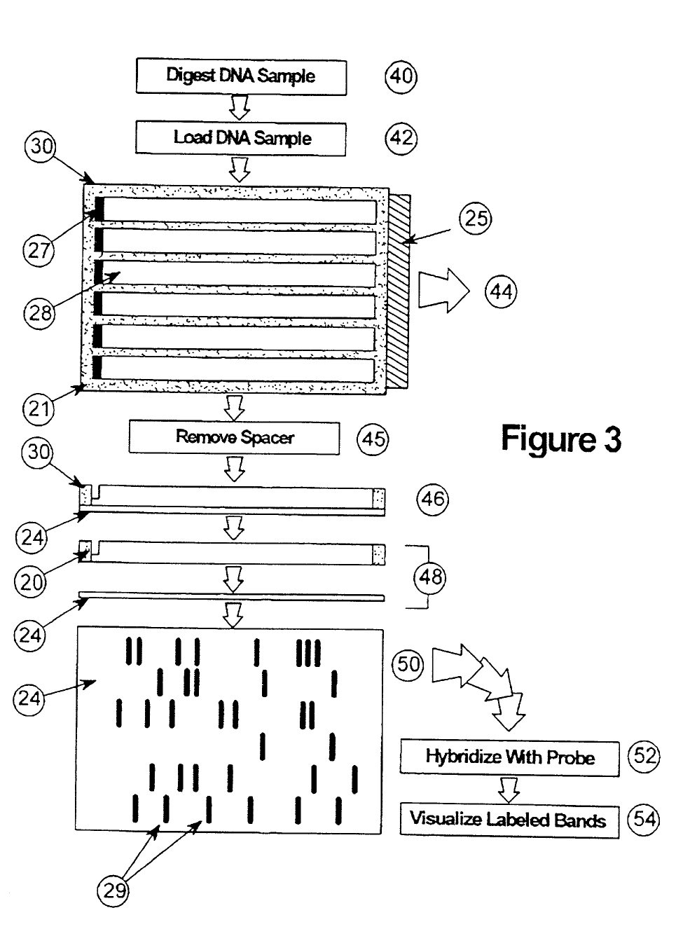

The porous polymer gel carrier is readily adapted for use with well known molecular blotting techniques, including the various types of Southern blotting technique which are widely used in the identification of specific DNA fragments. As schematically illustrated in FIG. 3, in Southern blot type analysis, the DNA material may be first digested with a selected restriction enzyme to create DNA fragments 40. The resulting DNA fragments may then be loaded 42 onto the sample wells 27 of an electrophoresis gel carrier 30 comprising a porous polymer layer 20, laminate layer 24, non porous spacer sheet 25 and a selected concentration of polyacrylimide, agarose gel or other separation material. In this particular embodiment, the laminate layer 24 is a nylon transfer membrane. The laminate layer 24 illustrated in FIG. 3 may also be constructed of another commonly used transfer materials such as nitrocellulose or PVDF.

After the sample is loaded 42, the electrophoresis gel carrier 30 may be placed in a conventional horizontal electrophoresis device and current is applied from the top edge of the gel carrier to the bottom edge in an orientation similar to that illustrated in FIG. 1a 44. At the conclusion of this initial electrophoresis run, the carrier is removed from the horizontal device and the spacer sheet may be removed 45. The gel carrier may then be placed between a second set of negative and positive electrodes for electroblotting 46. As illustrated in FIG. 1b, in electroblotting the electrodes may be located perpendicular to the gel carrier's horizontal plane. A second current may then be applied from the upper 21 to the lower 22 surface of the electrophoresis gel carrier 30. The component molecules of the sample are thus transferred from the separation channels 28 of the upper layer 21 to the transfer membrane 24. The transfer membrane 24 may then be removed from the electrophoresis gel carrier 30. The samples bound to the transfer membrane 24 may then be allowed to react or hybridize 52 with a probe or reagent that has been bonded to the transfer membrane 24. The resulting electrophoresis bands 29 may be then examined and visualized 54 using conventional optical or ultraviolet illuminating means.

Although the Southern blotting technique is specific for DNA molecules, it will apparent to one skilled in the art that the method and apparatus is readily adaptable to other nucleic acid blotting techniques including variations of the Northern blotting technique that is used for analyzing and identifying specific RNA fragments.

The combination of the porous polymer layer 20 with an additional laminate layer 24 provides a number of novel improvements over existing methods and devices. In existing methods of electrophoresis and molecular blotting, the conventional transfer of a gel to the transfer membrane may involve multiple manual transfers and manipulations of the gel. For example, in conventional molecular blotting techniques, following electrophoresis, the gel is removed from the transparent plates. A sandwich of filter paper soaked in buffer, a gel, a transfer membrane and an additional layered filter paper soaked in buffer is created. The resulting gel sandwich may then be manually blotted or placed between opposing electrodes in an electroblotting device. This often involves a significant amount of skillful and time consuming manipulation of the fragile gel material. Moreover, any error in technique or reagent preparation may seriously compromise the subsequent analysis.

In some embodiments, the porous polymer layer 30 may be combined with other porous polymer layers for electrophoresis or electroblotting by manual, robotic or automatic means. In such embodiments, multiple porous polymer gel carriers may be combined in a vertical or horizontal orientation relative to the opposing electrodes. The individual porous polymer gel carriers may also be separated by cover plates or by an additional laminate layer that may be attached to the upper surface 21 of each porous polymer carrier 30.

The method and apparatus is also readily adapted to the analysis of proteins. The Western blotting technique is commonly used to identify specific proteins or protein constituents in complex extracts of tissues and cells. This technique is widely used in diagnostic testing, blood screening, vaccine testing and other types of immunologic screening. In Western type blotting, protein mixtures that have been separated by gel electrophoresis are transferred to a solid support material, typically nitrocellulose or nylon.

The transfer to a solid support material facilitates the use of labeled protein probes such as antibodies. Antibodies bind specifically to certain antigen targets. Each antibody molecule recognizes its complementary antigen with great specificity. In Western type blotting, labeled antibody probes may be used to detect specific antigen targets that are contained in the protein mixture that has been blotted from the gel to the transfer material. The labeled antibody binds specifically to the corresponding antigen portion of the sample protein. The resulting labeled sample bands locate the specific antigen.

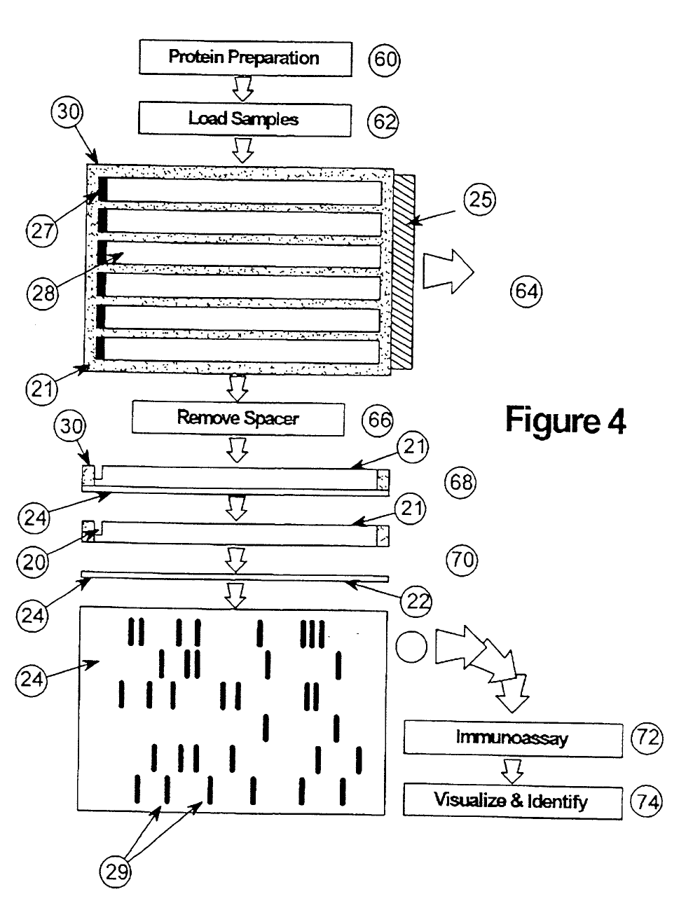

The use of the present invention for electrophoresis and Western type blotting is illustrated in FIG. 4. In this embodiment, the protein sample is prepared for analysis 60 by conventional methods that are well known in the art. The resulting samples are loaded 62 on to the sample well 27 of a separation channel 28 of the porous polymer gel carrier 30. As shown in FIG. 4, the laminate layer 24 of the porous polymer gel carrier is a nylon transfer membrane. In the illustrated embodiment, a second non-porous and non-conductive freely removable spacer sheet 25 of polyethylene or other suitable material may be inserted between the gel containing porous polymer layer 20 and the laminate layer 24. The nylon transfer membrane 24 may be attached to the edge of the porous polymer layer 20 by heat, pins or other adhesive means. Because the freely removable spacer sheet 25 is narrower in width than then the porous polymer layer 20, the laminate layer 24 may be firmly attached to the bottom surface 22 of the porous polymer layer 20. The gel carrier 30 may then be used in a horizontal or vertical electrophoresis device 64. In the electrophoresis phase 64, illustrated in FIG. 4, the current is applied in the direction of the arrow.

Upon completion of the electrophoresis phase 64 the samples may be analyzed by various molecular blotting methods. The non-porous spacer sheet 25 may be removed 66. After removing the non-porous spacer sheet 25, the samples contained in the separation channels 28 may be blotted 68 down on the nylon transfer membrane 24 using a conventional blotting techniques or by the electroblotting method that is generally illustrated in FIG. 1b and is previously discussed herein. The nylon transfer layer 24 can then be removed 70 from the polymer layer 20 for further analysis. The results may be visualized by conventional techniques 74. Alternatively, the transfer membrane may also be stored for future analysis.

In yet another embodiment of this invention, the laminate transfer layer 24 may be pretreated with a selected molecular probe, antibody or hybridizing reagent. According to this embodiment, during and after electroblotting, the samples may be allowed to react with a labeled probe or reagent that has been prebonded to the laminate layer 24. The results may then be examined using an appropriate optical, or ultraviolet illumination source or other conventional detection methods.

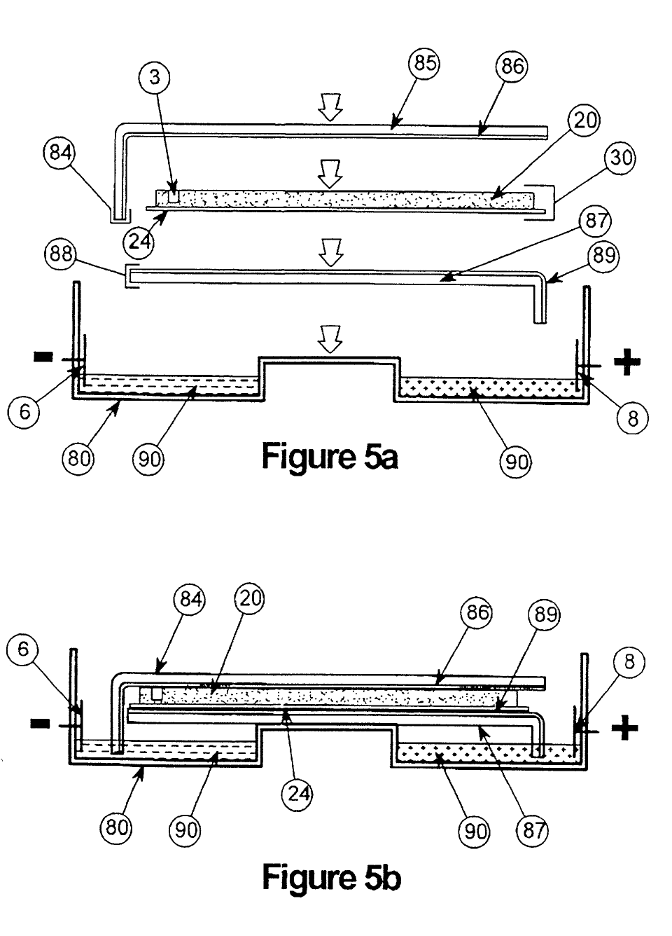

The modular and flexible nature of the electrophoresis gel carrier 30 also means that it may be readily adapted for use in existing horizontal electrophoresis units using may be readily adapted to perform electroblotting using a novel combination of electrophoresis paddles. As illustrated in FIG. 5, a conventional horizontal electrophoresis unit 80 may contain a buffer containing chamber 82 or chambers. The negative 6 and positive 8 electrodes are located at opposing ends of the buffer reservoir 90.

The specific improvement comprises a pair of “L”-shaped electrophoresis paddles 84, 88 that are adapted for use in such commercially available horizontal electrophoresis device 80. As schematically illustrated in FIG. 5, the upper electrophoresis paddle 84 is constructed of a suitable non-conductive material such as plastic. The bottom surface 86 of the upper electrophoresis paddle 84 may be constructed of stainless steel or of a similar material. The lower electrophoresis paddle 86 is constructed of suitable non-conducting material, such as plastic. The top portion 89 of the lower electrophoresis paddle 86 may incorporate a suitable non-corroding and conductive material such as platinum wire or mesh.

As illustrated in FIG. 5a, a sample containing porous polymer gel carrier 30 may be then placed between the opposing electrophoresis paddles 84, 88. The resulting paddle gel carrier assembly then be placed in the buffer reservoir 90 so that the shorter portion of each “L” shaped paddle may immersed in the buffer solution and capable of conducting the electric current. As shown in FIG. 5b, the use of the electrophoresis paddles 84, 88 permits a standard horizontal electrophoresis unit to readily use the porous polymer gel carrier 30 for electroblotting while retaining all of the safety features of an original electrophoresis device. In some applications, it may be desirable to use one or more cover plates 4, 5 between the gel carrier 30 and the electrophoresis paddles 84, 88. This permits existing electrophoresis devices to be used for electrophoresis and electroblotting.

The present invention affords a simple and economical way to prepare, use and distribute pre-cast electrophoresis separation and blotting materials. The pre-cast nature of the electrophoresis gel carrier 30 may eliminate the need for complicated and potentially inconsistent preparation by individual users. The pre-casting and distribution of the separation material also permits a degree of quality control and standardization that is not possible with existing methods and materials.

The invention is also readily adapted to the prepackaging of standardized electrophoresis and blotting reagents. The resulting simplification and standardization will facilitate the commercial, diagnostic and forensic use of these techniques. As described, the size and shape of the electrophoresis gel carrier 30 and the width and depth of sample channels 28 can be readily adapted to a full spectrum of uses. The separation media containing porous polymer gel carrier can be easily pre-packaged in shrink wrap or other suitable packaging material. Furthermore, because the porous polymer gel carrier matrix 30 may be conveniently transported, stored and utilized, many of the risks that are otherwise inherent in the shipping, handling and storage of fragile electrophoresis materials are dramatically reduced. The porous polymer gel carrier also facilitates storage of the electrophoresis material for future analysis.

While preferred embodiments of the present invention have been described, it is to be understood that the embodiments described are illustrative only and the scope of the invention is to be defined solely to the appended claims when accorded a full range of equivalents, any modifications naturally occurring to those in the art from a perusal hereof.

I claim the following:

1. An electrophoresis gel carrier capable of being inserted into an electrophoresis device comprising:
(a) a porous polymer layer having opposing top and bottom surface that are separated by opposing sides;
(b) a non-porous laminate layer attached directly to said bottom surface of said porous polymer layer, and
(c) one or more separation channels oriented generally parallel to an axis of selected said porous polymer layer, wherein each said separation channel has a depth equal to the thickness of said porous polymer layer and is capable of holding a selected separation material.

2. The electrophoresis gel carrier of claim 1 wherein said porous polymer layer is constructed of polyethylene, reticulated polyurethane or any open cell nonconductive material.

3. The electrophoresis gel carrier of claim 2 wherein said separation channel contains a separation material selected from the group of polyacrylimide or agarose gel.

4. The electrophoresis gel carrier of claim 3 wherein said non-porous laminate layer is freely removable from said porous polymer layer.

5. The electrophoresis gel carrier of claim 4 wherein said laminate layer is a nylon transfer membrane.

6. The electrophoresis gel carrier of claim 5 wherein said laminate layer is separated from said porous polymer layer by a freely removable non-porous and nonconductive spacer material.

7. The electrophoresis gel carrier of claim 6 wherein said laminate layer is pretreated with a reagent that is capable of selectively binding to a specified chemical component.

8. A method for fabricating a porous polymer electrophoresis gel carrier comprising the following steps:
(a) preparing a liquid solution of a porous separation material;
(b) preparing a layer of porous polymer material having:
(i) opposing top and bottom surfaces and opposing sides of equal thickness;
(ii) one or more separation channels that are oriented generally parallel to selected axis of said porous polymer layer wherein each said separation channel extends through the entire thickness of said porous polymer layer and is adapted to hold said separation material;
(c) attaching a laminate layer directly to said bottom surface of said porous polymer layer;
(d) filling each said separation channel with said porous separation material; and
(e) providing a sample loading well in each said separation channel.

9. The method of claim 8 wherein said porous polymer material is selected from the group comprising polyethylene, reticulated polyurethane, or any open cell nonconductive material.

10. The method of claim 9 wherein said laminate layer is an optically transparent material.

11. The method of claim 10 wherein said porous separation material is selected from the group of agarose or polyacrylamide gel.

12. The method of claim 11 wherein said separation medium is a combination of agarose and polyacrylamide.

13. The method of claim 8 where the said opposing sides are sealed by heat or other sealant means.

14. A porous polymer gel carrier for electrophoresis and blotting comprising:
(a) a polymer layer comprising a porous polymer material;
(b) a porous separation material;
(c) one or more separation channels configured to hold said porous separation material wherein each said separation channel is oriented generally parallel to a selected axis of said porous polymer layer and passes entirely between the upper and lower surface of said porous polymer layer; and
(d) a laminate layer of non-porous material attached directly to a lower surface of said porous polymer layer.

15. The porous polymer gel carrier of claim 14 wherein each said separation channels contains a electrophoresis separation material selected from the group of polyacrylimide or agarose gels.

16. The porous polymer gel carrier of claim 15 wherein said laminated layer is a nylon transfer membrane.

17. The porous polymer gel carrier of claim 16 wherein said laminate layer is separated from said porous polymer layer by a freely removable non-porous and non-conductive spacer strip.

18. The porous polymer gel carrier of claim 17 wherein said laminate layer is pretreated with a reagent that is capable of selectively binding to a specified chemical component.

19. A method of electrophoresis and blotting using a porous polymer gel carrier comprising the following steps:
(a) preparing a porous polymer gel carrier comprising:
(i) a polymer layer of porous polymer material having opposing top and bottom surfaces and opposing sides of equal thickness;
(ii) a porous separation material;
(iii) providing in said polymer layer one or more separation channels configured to hold said porous separation material and oriented in parallel to a selected axis of said top and bottom surface of the polymer layer;
(iv) an optically transparent non-porous laminate layer directly to the bottom surface of said polymer layer.
(b) filling each said separation channel with said porous separation material;
(c) providing one or more loading wells for each said separation channel;
(d) loading a predetermined amount of a sample material into each said sample channels;
(e) placing said porous polymer gel carrier into an electrophoresis means that is adapted to receive said porous polymer gel carrier;
(f) applying a predetermined amount of current to said porous polymer gel;
(g) removing said porous polymer gel carrier from said electrophoresis device; and
(h) transferring said sample said from separation channel to said laminate layer by blotting.

20. The method of claim 20 wherein the said porous polymer gel carrier has a freely removable porous non-conducting spacer sheet between said porous polymer layer and said laminate layer.

21. The method of claim 20 wherein the laminate layer is a nylon transfer membrane.

22. The method of claim 21 wherein the laminate layer is pretreated with a selected molecular probe capable of selectively binding to a specific chemical component.

23. A porous polymer gel carrier which comprises:
(a) a porous polymer layer;
(b) a porous separation material such as agarose or polyacrylimide;
(c) a non-porous laminate layer attached to the bottom surface of said porous polymer layer.
(d) one or more separation channels oriented parallel to an axis of the porous polymer layer wherein said separation channels contain said electrophoresis separation material; and
(e) a freely removable non-porous, non-conducting spacer sheet placed between said bottom surface of said porous polymer layer and said laminate layer.

24. The porous polymer gel carrier of claim 23 wherein said sample channels contain an electrophoresis separation material that is a porous separation material, other than polyacrylimide or agarose.

25. A porous polymer gel carrier constructed by a process comprising:
(a) preparing a polymer layer from a porous polymer material wherein said first layer has opposing top and bottom and opposing sides of equal depth;
(b) providing in said first layer one or more substantially parallel separations channels wherein each said separation channel is configured to contain a separation material and is oriented parallel to an axis of the first polymer layer wherein each sample channel passes through the entire thickness of the porous polymer layer;
(c) heat sealing said sides of the first layer;
(d) attaching a laminate layer directly to the bottom surface of the porous polymer layer;
(e) filling each said sample channel with said electrophoresis separation material in liquid state; and
(f) allowing the said electrophoresis separation material to solidify.

26. The porous polymer gel carrier of claim 26 wherein the laminate layer is a nylon transfer membrane.

27. The porous polymer gel carrier of claim 27 wherein the nylon transfer membrane is pretreated with a chemical probe that is capable of selectively bonding with a specific chemical constituent of a sample.

28. A porous polymer carrier assembly comprising:
(a) A porous polymer gel carrier comprising:
(i) a porous polymer layer containing one or more generally parallel separation channels containing an electrophoresis separation material;
(ii) a second laminate layer bonded directly to the bottom surface of the porous polymer layer.
(b) a pair of electrophoresis paddles configured to hold said porous polymer gel carrier comprising:
(i) a first electrophoresis paddle configured to hold said porous polymer gel carrier wherein a first end of said first electrophoresis paddle is in contact with a quantity of a buffer solution; and
(ii) a second electrophoresis paddle configured to hold said gel carrier wherein a first end of said second electrophoresis paddle is capable of contacting a quantity of a buffer; and
(c) a container configured to hold a first and a second quantity of buffer solution, wherein said first quantity of buffer solution is an contact with said first electrophoresis paddle and said second quantity of buffer solution is in contact with said second electrophoresis paddle.

29. A device for electrophoresis comprising a pair of opposing electrodes, an electrophoresis separation media, a reservoir, and a buffer solution wherein the improvement comprises:
(a) a porous polymer gel carrier constructed of a porous polymer layer that is bonded directly to a lower laminate layer and which contains one or more separation channels containing an electrophoresis separation material;
(b) a pair of electrophoresis paddles that are adapted to be placed in a reservoir containing a buffer solution and adapted to transmit current between opposing electrodes of an electrophoresis apparatus in a direction that is perpendicular to the plane of said porous polymer gel carrier wherein said pair of electrophoresis paddles being further adapted to hold said porous polymer gel carrier.
